(19) United States
(12) Patent Application Publication
(10) Pub. No.: US 2004/0106110 A1
(43) Pub. Date: Jun. 3, 2004

(54) Preparation of polynucleotide arrays

(75) Inventors: Shankar Balasubramanian, Nr Saffron Walden (GB); David Klenerman, Nr Saffron Walden (GB); Colin Barnes, Nr Saffron Walden (GB); Mark Allen Osborne, Nr Saffron Walden (GB)

Correspondence Address: KLAUBER & JACKSON, 411 HACKENSACK AVENUE, HACKENSACK, NJ, 07601

(73) Assignee: Solexa, Ltd.

(21) Appl. No.: 10/153,267

(22) Filed: May 22, 2002

Related U.S. Application Data
(63) Continuation-in-part of application No. PCT/GB02/00438, filed on Jan. 30, 2002.
Application No. PCT/GB02/00438 is a continuation-in-part of application No. 09/771,708, filed on Jan. 30, 2001.
Application No. PCT/GB02/00438 is a continuation-in-part of application No. 09/771,708, filed on Jan. 30, 2001.
Application No. 09/771,708 is a continuation-in-part of application No. PCT/GB99/02487, filed on Jul. 30, 1999.

(30) Foreign Application Priority Data
Oct. 16, 1998 (GB) GB9822670.7
Jun. 30, 1999 (EP) EP98306094.8

(51) Int. Cl. C12Q 1/68; C12M 1/34

(52) U.S. Cl. 435/6; 435/287.2

(57) ABSTRACT

A device comprising an array of molecules immobilised on a solid surface is disclosed, wherein the array has a high density of relatively short molecules and relatively long polynucleotides immobilised on the surface of a solid support, wherein the polynucleotides are at a density that permits individual resolution of those parts thereof that extend beyond the relatively short molecules.

### RELATED APPLICATIONS

This application is a continuation-in-part of International Application No. PCT/GB02/00438, filed Jan. 30, 2002, which designated the United States and will be published in English, and which, along with the present application, is a continuation-in-part of application Ser. No. 09/771,708, filed Jan. 30, 2001, which is a continuation-in-part of International Application No. PCT/GB99/02487, which designated the United States and was filed on Jul. 30, 1999, published in English, which in turn claims priority to European App. No. 1998306094.8, filed on Jul. 30, 1998, and also Great Britain App. No. 199822670.7, filed Oct. 16, 1998. The entire teachings of the above applications are incorporated herein by reference.

### FIELD OF THE INVENTION

This invention relates to fabricated arrays of polynucleotides, and to their analytical applications.

### BACKGROUND

Advances in the study of molecules have been led, in part, by improvement in technologies used to characterise the molecules or their biological reactions. In particular, the study of nucleic acids, DNA and RNA, has benefited from developing technologies used for sequence analysis and the study of hybridisation events.

An example of the technologies that have improved the study of nucleic acids, is the development of fabricated arrays of immobilised nucleic acids. These arrays typically consist of a high-density matrix of polynucleotides immobilised onto a solid support material. Fodor et al., Trends in Biotechnology (1994) 12:19-26, describes ways of assembling the nucleic acid arrays using a chemically sensitised glass surface protected by a mask, but exposed at defined areas to allow attachment of suitably modified nucleotides. Typically, these arrays may be described as “many molecule” arrays, as distinct regions are formed on the solid support comprising a high density of one specific type of polynucleotide.

An alternative approach is described by Schena et al., Science (1995) 270:467-470, where samples of DNA are positioned at predetermined sites on a glass microscope slide by robotic micropipetting techniques. The DNA is attached to the glass surface along its entire length by non-covalent electrostatic interactions. However, although hybridisation with complementary DNA sequences can occur, this approach may not permit the DNA to be freely available for interacting with other components such as polymerase enzymes, DNA-binding proteins etc.

WO-A-96/27025 is a general disclosure of single molecule arrays. Although sequencing procedures are disclosed, there is little description of the applications to which the arrays can be applied. There is also only a general discussion on how to prepare the arrays.

### SUMMARY OF THE INVENTION

According to the present invention, a device comprises a high density array of single polynucleotide molecules, comprising relatively short molecules and relatively long polynucleotides immobilised on the surface of a solid support, where the relatively long polynucleotides are at a density that permits individual resolution and/or interrogation of those parts that extend beyond the relatively short molecules. The device can be any device that comprises this array, including, but not limited to, a sequencing machine or genetic analysis machine. In this aspect, the relatively short molecules help to control the density of the relatively long polynucleotides, providing a more uniform array of single polynucleotide molecules, thereby improving imaging. The relatively short molecules can also prevent non-specific binding of reagents to the solid support, and therefore reduce background interference. For example, in the context of a polymerase reaction to incorporate nucleoside triphosphates onto a strand complementary to a relatively long polynucleotide, the relatively short molecules prevent the polymerase and nucleosides from attaching to the solid support surface, which may otherwise interfere with the imaging process.

The relatively short molecules can also ensure that each relatively long polynucleotide is maintained upright, preventing the polynucleotides from interacting lengthwise with the solid support, which may otherwise prevent efficient interaction with a reagent, e.g., a polymerase. This can also prevent the fluorophore being quenched by the surface and therefore lead to more accurate imaging of the single polynucleotide molecules.

As used herein, the term “array” refers to a population of polynucleotide molecules that are distributed over a solid support; preferably, these polynucleotides are spaced at a distance from one another sufficient to permit the individual resolution of the polynucleotides.

“Relatively long polynucleotides”, “long polynucleotides”, “and single polynucleotide molecules”, are used interchangably herein. “Relatively short molecules”, “short molecules”, “relatively small molecules” and “small molecules”, are also used interchangably herein. In the context of the present invention, the terms “relatively short” and “relatively long” should be interpreted to mean that the portion of at least a subset of the “relatively long” polynucleotides that is not used for attachment to the substrate or to a linker molecule(s) attached to the substrate, is physically longer than that of the “relatively short” molecules when the relatively long polynucleotides and the relatively short molecules are arrayed. In general, the relatively long polynucleotides can be one nucleotide (or one nucleotide pair, if the polynucleotide is double stranded) or greater in length than the relatively short molecules. That is, the relatively long polynucleotides are longer, with respect to the distance from the planar surface of the solid support, than the relatively short molecules. The length of the long polynucleotides can be 50 to 10,000 nucleotides in length, preferably 100 to 1000 nucleotides in length. If the relatively short molecules are not polynucleotides, then the relatively long polynucleotides are at least the equivalent physical distance of one nucleotide longer (or one nucleotide pair, if the polynucleotide is double stranded) than the relatively short molecules. The term “relatively long” also encompasses polynucleotides which extend above the relatively short molecules in an array format where the relatively long polynucleotides are distributed on the solid support at a density of about 106 to about 10⁹ polynucleotides per cm², and where the relatively short molecules are distributed at a density greater than about 10⁸ to about 10¹⁴ molecules per cm². In general, the surface of the substrate is engineered so that the short molecules display a hydrophilic group from the surface. The relatively short molecules can therefore be silanes, amino acids, an acid, phosphate, thiophosphate, sulfate, thiol, hydroxyl or polyol, etc. and may include polyethers such as PEG. The types of molecules used will also depend on the surface chemistry used to attach the long molecules to the surface.

As used herein, the term “single polynucleotide molecule” refers to one polymeric molecule of a nucleic acid sequence. Thus, an array feature or address corresponding to a single relatively long polynucleotide consists of one polynucleotide molecule immobilized onto a solid support. The immobilized single polynucleotide molecule can be single- or double-stranded, or have both single-stranded portions and double-stranded portions. For example, it can include a hairpin. In one embodiment, the single polynucleotide molecule is both single-stranded and double-stranded. This is in contrast to the arrays of the prior art, in which a given address typically comprises a plurality of copies (e.g., 10 or more) of a given nucleic acid molecule, often thousands of copies or more. The term “single molecule” is also used herein to distinguish from high density multi-molecule (polynucleotide) arrays in the prior art, which may comprise distinct clusters of many polynucleotides of the same type. As used herein, at least some (e.g., 10 or more) of the addresses in the array are intended to be populated by only one polynucleotide molecule.

“Solid support”, as used herein, refers to the material to which the relatively long polynucleotides and relatively short molecules are attached. Suitable solid supports are available commercially, and will be apparent to the skilled person. The supports can be manufactured from materials such as glass, ceramics, silica and silicon. Supports with a gold surface may also be used. The supports usually comprise a flat (planar) surface, or at least a structure in which the polynucleotides to be interrogated are in approximately the same plane. Alternatively, the solid support can be non-planar, e.g., a microbead. Any suitable size may be used. For example, the supports might be on the order of 1-10 cm in each direction.

The term “individually resolved by optical microscopy” is used herein to indicate that, when visualised, it is possible to distinguish at least one polynucleotide on the array from its neighbouring polynucleotides using optical microscopy methods available in the art. Visualisation may be effected by the use of reporter labels, e.g., fluorophores, the signal of which is individually resolved. As used herein, the term “interrogate” means contacting one or more of the relatively long polynucleotides with another molecule, e.g., a polymerase, a nucleoside triphosphate, a complementary nucleic acid sequence, wherein the physical interaction provides information regarding a characteristic of the arrayed polynucleotide. The contacting can involve covalent or non-covalent interactions with the other molecule. As used herein, “information regarding a characteristic” means information regarding the sequence of one or more nucleotides in the polynucleotide, the length of the polynucleotide, the base composition of the polynucleotide, the Tₘ of the polynucleotide, the presence of a specific binding site for a polypeptide or other molecule, the presence of an adduct or modified nucleotide, or the three-dimensional structure of the polynucleotide.

As used herein, the term “portion that is immobilized by bonding to the surface” refers to the nucleotide or nucleotides of an immobilized single polynucleotide molecule that is or are either directly involved in linkage to the solid substrate or an intermediate linker molecule (which is then bound to the substrate), or, because of their proximity to the point of immobilization, are not physically accessible to be capable of interrogation (e.g., to serve as a template or substrate for the primer extension activity of a nucleic acid polymerase enzyme). It is preferred that polynucleotides be immobilized by either their 5′ end or their 3′ end, but polynucleotides can also be immobilized via one or more internal nucleotides.

As used herein, the term “portion that is capable of interrogation” refers to that portion of an immobilized polynucleotide molecule that is physically accessible to a physical interaction with another molecule or molecules, the interaction of which provides information regarding a characteristic of the arrayed polynucleotide as defined herein. Generally, the “portion of an immobilized single polynucleotide molecule that is capable of interrogation” is that part which is not the “portion that is immobilized by covalent bonding to the surface” as that term is defined herein.

In one aspect of the invention, the device comprises a high density array of a plurality of first molecules, i.e., the relatively short molecules, and a plurality of second polynucleotides, i.e., the relatively long polynucleotides, immobilised on the surface of a solid support, where each molecule of at least a subset of the plurality of first molecules is shorter in length than the length of each of the second polynucleotide of at least a subset of the plurality of second polynucleotides such that the second polynucleotides are of a length and at a density that permits individual resolution of at least two of the second polynucleotides of the subset. “Plurality” is used to mean that multiple short molecules and multiple long polynucleotides are placed on the array. The short molecules can be of all the same type, or of multiple, i.e., different, types. The long polynucleotides will also generally be of multiple types, and can all be different from each other. The long polynucleotides can also be of different lengths relative to each other, e.g., some of the polynucleotides may be 100 nucleotides in length, while others may be 120 nucleotides in length. By saying that each molecule of “at least a subset” of the plurality of first molecules is shorter in length than the length of each of the second polynucleotide of “at least a subset” of the plurality of second polynucleotides, is meant that one practicing the invention has arrayed polynucleotides that are intended to be physically longer (in that portion of the relatively long polynucleotide that is not used for attachment to the substrate or to a linker molecule(s) attached to the substrate) than the short molecules, but due to breakage of the polynucleotides or binding of short molecules to each other, or some other occurrence, not every individual polynucleotide may be longer than every short molecule.

According to a second aspect of the invention, a method for the production of an array of polynucleotides which are at a density that permits individual resolution, comprises arraying on the surface of a solid support, a mixture of relatively short molecules and relatively long polynucleotides, wherein the short molecules are arrayed in an amount in excess of the polynucleotides. By “in excess” is meant that, in such an embodiment, the small molecules are at a density of from 108 to 10¹⁴ molecules/cm², more preferably greater than 10¹² molecules/cm², whereas the long polynucleotides are at a density of 10⁶ to 10⁹ polynucleotides per cm², preferably 10⁷ to 10⁹ polynucleotides per cm².

In another aspect, only a minor proportion of the short molecules that are arrayed at high density on the solid support comprise a group that reacts with the polynucleotides; the majority are non-reactive. In general “a minor proportion” means that reactive and non-reactive molecules exist on the substrate in a ratio of about 1/10 to about 1/1,000,000, preferably about 1/10 to about 1/10,000.

For example, the short molecules can be mixed silanes, a minor proportion of which are reactive with a functional group on the polynucleotides, and the remaining silanes are unreactive and form the array of short molecules on the device. Therefore, controlling the concentration of the minor proportion of short molecules also controls the density of the polynucleotides.

The arrays of the present invention comprise what are effectively single analysable polynucleotides. This has many important benefits for the study of the polynucleotides and their interaction with other biological molecules. In particular, fluorescence events occurring on each polynucleotide can be detected using an optical microscope linked to a sensitive detector, resulting in a distinct signal for each polynucleotide.

When used in a multi-step analysis of a population of single polynucleotides, the phasing problems (loss of syncronization) that are encountered using high density (multi-molecule) arrays of the prior art, can be reduced or removed. Therefore, the arrays also permit a massively parallel approach to monitoring fluorescent or other events on the polynucleotides. Such massively parallel data acquisition makes the arrays extremely useful in a wide range of analysis procedures which involve the screening/characterising of heterogeneous mixtures of polynucleotides.

The preparation of the arrays requires only small amounts of polynucleotide sample and other reagents, and can be carried out by simple means.

### BRIEF DESCRIPTION OF THE DRAWINGS

FIGS. 1a and b are images of a single polynucleotide array, where single polynucleotides are indicated by the detection of a fluorescent signal generated on the array.

### DETAILED DESCRIPTION

The single polynucleotide array devices of the present invention are fabricated to include a “monolayer” of relatively short molecules that coat the surface of a solid support material and provide a flexible means to control the density of the single polynucleotides and optionally to prevent non-specific binding of reagents to the solid support.

The single polynucleotides immobilised onto the surface of a solid support should be capable of being resolved by optical means. This means that, within the resolvable area of the particular imaging device used, there must be one or more distinct signals, each representing one polynucleotide. Typically, the polynucleotides of the array are resolved using a single molecule fluorescence microscope equipped with a sensitive detector, e.g., a charge-coupled device (CCD). Each polynucleotide of the array may be imaged simultaneously or, by scanning the array, a fast sequential analysis can be performed.

The long polynucleotides of the array are typically DNA or RNA, although nucleic acid mimics, e.g., PNA or 2′-O-methyl-RNA, are within the scope of the invention. The long polynucleotides are formed on the array to allow interaction with other molecules. It is therefore important to immobilise the long polynucleotides so that the portion of the long polynucleotide not physically attached to solid support is capable of being interrogated. In some applications all the long polynucleotides in the single array will be the same, and may be used to capture molecules that are largely distinct. In other applications, the long polynucleotides on the array may all, or substantially all, be different, e.g., less than 50%, preferably less than 30% of the long polynucleotides will be the same.

The term “interrogate” is used herein to refer to any interaction of the arrayed long polynucleotide with any other molecule, e.g., with a polymerase or nucleoside triphosphate or a complementary nucleic acid sequence.

The density of the arrays is not critical. However, the present invention can make use of a high density of single long polynucleotides, and these are preferable. For example, arrays with a density of 10⁶-10⁹ long polynucleotides per cm² may be used. Preferably, the density is at least 10⁷/cm² and typically up to 10⁹/cm². These high density arrays are in contrast to other arrays which may be described in the art as “high density” but which are not necessarily as high and/or which do not allow single molecule resolution.

The shorter molecules will typically be present on the array at much higher density than the relatively long polynucleotides, to coat the surface of the solid support not occupied by the relatively long polynucleotides. The shorter molecules may therefore be brought into contact with the solid support at an excess concentration. Preferably, the small molecules are at a density of from 108 to 1014 molecules/cm², more preferably greater than 10¹² molecules/cm².

Using the methods and device of the present invention, it may be possible to image at least 106-109, preferably 107 or 108 long polynucleotides/cm². Fast sequential imaging may be achieved using a scanning apparatus; shifting and transfer between images may allow higher numbers of polynucleotides to be imaged.

The extent of separation between the individual polynucleotides on the array will be determined, in part, by the particular technique used to resolve the individual polynucleotide.

Apparatus used to image molecular arrays are known to those skilled in the art. For example, a confocal scanning microscope may be used to scan the surface of the array with a laser to image directly a fluorophore incorporated on the individual polynucleotide by fluorescence. Alternatively, a sensitive 2-D detector, such as a charge-coupled device, can be used to provide a 2-D image representing the individual polynucleotides on the array. “Resolving” single polynucleotides on the array with a 2-D detector can be done if, at 100× magnification, adjacent polynucleotides are separated by a distance of approximately at least 250 nm, preferably at least 300 nm and more preferably at least 350 nm. It will be appreciated that these distances are dependent on magnification, and that other values can be determined accordingly, by one of ordinary skill in the art.

Other techniques such as scanning near-field optical microscopy (SNOM) are available which are capable of greater optical resolution, thereby permitting more dense arrays to be used. For example, using SNOM, adjacent polynucleotides may be separated by a distance of less than 100 nm, e.g., 10 nm. For a description of scanning near-field optical microscopy, see Moyer et al., Laser Focus World (1993) 29(10).

An additional technique that may be used is surface-specific total internal reflection fluorescence microscopy (TIRFM); see, for example, Vale et al., Nature (1996) 380:451-453). Using this technique, it is possible to achieve wide-field imaging (up to 100 μm×100 μm) with single molecule sensitivity. This may allow arrays of greater than 10⁷ resolvable polynucleotides per cm² to be used.

Additionally, the techniques of scanning tunnelling microscopy (Binnig et al., Helvetica Physica Acta (1982) 55:726-735) and atomic force microscopy (Hansma et al., Ann. Rev. Biophys. Biomol. Struct. (1994) 23:115-139) are suitable for imaging the arrays of the present invention. Other devices which do not rely on microscopy may also be used, provided that they are capable of imaging within discrete areas on a solid support.

The devices according to the invention comprise immobilised polynucleotides and other immobilised molecules. The other molecules are relatively short compared to the polynucleotides and are used to control the density of the polynucleotides. They may also prevent non-specific attachment of reagents, e.g., nucleoside triphosphates, with the solid support, thereby reducing background interference. In one embodiment, the shorter molecules are also polynucleotides. However, other molecules may be used, e.g., peptides, proteins, polymers or synthetic chemicals, as will be apparent to the skilled person and depending on the application to which the array will be used. The preferred molecules are organic molecules that contain groups that can react with the surface of a solid support.

Preparation of the devices may be carried out by first preparing a mixture of the relatively long polynucleotides and of the relatively short molecules. Usually, the concentration of the latter will be in excess of that of the long polynucleotides. By “in excess” is meant that the short molecules are at least 100-fold in excess of the long molecules. The mixture is then placed in contact with a suitably prepared solid support, to allow immobilisation to occur.

Single polynucleotides may be immobilised to the surface of a solid support by any known technique, provided that suitable conditions are used to ensure adequate separation. Density of the polynucleotide molecules may be controlled by dilution. The gaps between the polynucleotides can be filled in with short molecules (capping groups) that may be small organic molecules or may be polynucleotides of different composition. The formation of the array of individually resolvable “longer” polynucleotides permits interrogation of those polynucleotides that are different from the bulk of the molecules.

Immobilisation may be by specific covalent or non-covalent interactions. Covalent attachment is preferred. Immobilisation of a polynucleotide will be carried out at either the 5′ or 3′ position, so that the polynucleotide is attached to the solid support at one end only. However, the polynucleotide may be attached to the solid support at any position along its length, the attachment acting to tether the polynucleotide to the solid support; this is shown for the hairpin constructs, described below. The immobilised (relatively long) polynucleotide is then able to undergo interactions with other molecules or cognates at positions distant from the solid support. Immobilisation in this manner results in well separated long polynucleotides. The advantage of this is that it prevents interaction between neighbouring long polynucleotides on the array, which may hinder interrogation of the array.

Suitable methods for forming the devices with relatively short molecules and relatively long polynucleotides will be apparent to the skilled person, based on conventional chemistries. The aim is to produce a highly dense layer of the relatively short molecules, interspersed with the relatively large polynucleotides which are at a density that permits resolution of each single polynucleotide.

A first step in the fabrication of the arrays will usually be to functionalise the surface of the solid support, making it suitable for attachment of the molecules/polynucleotides. For example, silanes are known functional groups that have been used to attach molecules to a solid support material, usually a glass slide. The relatively short molecules and relatively long polynucleotides can then be brought into contact with the functionalised solid support, at suitable concentrations and in either separate or combined samples, to form the arrays.

In one preferred embodiment, the long polynucleotides and the short molecules each have the same reactive group that attaches to the solid support, or to an intermediary molecule.

In an alternative embodiment, the support surface may be treated with different functional groups, one of which is to react specifically with the relatively short molecules, and the other with the relatively long polynucleotides. Controlling the concentration of each functional group provides a convenient way to control the densities of the molecules/polynucleotides.

In a still further embodiment, the relatively short molecules are immobilised at high density onto the surface of the solid support. The molecules are capable of reacting with the polynucleotides (either directly or through an intermediate functional group) which can be brought into contact with the molecules at a suitable concentration to provide the required density. “Intermediate functional group” means any homo- or heterobifunctional crosslinking agent. The polynucleotides are therefore immobilised on top of the monolayer of molecules.

Those molecules that are not in contact with a polynucleotide may be reacted with a further molecule to block (or cap) the reactive site. This may be carried out before, during or after arraying the polynucleotides. The blocking (capping) group may itself be a relatively short polynucleotide.

In another embodiment, only a minor proportion of the short molecules that are arrayed at high density on the solid support comprise a group that reacts with the polynucleotides; the majority, e.g., 90% or greater, are non-reactive. For example, the short molecules can be mixed silanes, a minor proportion of which are reactive with a functional group on the polynucleotides, and the remaining silanes are unreactive and form the array of short molecules on the device. Therefore, controlling the concentration of the minor proportion of short molecules also controls the density of the polynucleotides.

In another embodiment, the short molecules may have been modified in solution prior to immobilisation on the array so that only a minor proportion contain a functional group that is capable of undergoing covalent attachment to a complementary functional group on the polynucleotides.

In a related embodiment, the relatively short molecules are polynucleotides, and appropriate concentrations of both relatively long and relatively short polynucleotides are reacted with a functional group and then arrayed on the solid support, or to an intermediate molecule bound to the solid support.

Suitable functional groups will be apparent to the skilled person. For example, suitable groups include: amines, acids, esters, activated acids, acid halides, alcohols, thiols, disulfides, olefins, dienes, halogenated electrophiles, thiophosphates and phosphorothioates. It is preferred if the group contains a silane.

The relatively small molecules may be any molecule that can provide a barrier against non-specific binding to the solid support.

Suitable small molecules may be selected based on the required properties of the surface and the existing functionality.

In a preferred embodiment, the molecules are silanes of type RₙSiX₍₄₋ₙ₎ (where R is an inert moiety that is displayed on the surface of the solid support and X is a reactive leaving group of type Cl or O-alkyl). The silanes include tetraethoxysilane, triethoxymethylsilane, diethoxydimethylsilane or glycidoxypropyltriethoxysilane, although many other suitable examples will be apparent to the skilled person.

In an embodiment of the invention, the short molecules act as surface blocks to prevent random polynucleotide association with the surface of the solid support. Molecules therefore require a group to react with the surface (which will preferably be the same functionality as used to attach the polynucleotide to the surface) and an inert group that will be defined by the properties required on the surface. In an embodiment, the surface is functionalised with an epoxide and the small molecule is glycine, although other compounds containing an amine group would suffice.

It is also preferred if the small molecule is hydrophilic and repels binding of anions. The molecule therefore may be acid, phosphate, sulfate, hydroxyl or polyol and may include polyethers such as PEG.

In one embodiment, the relatively short molecules are polynucleotides. These may be prepared using any suitable technique, including synthetic techniques known in the art. It may be preferable to use short polynucleotides that are immobilised to the solid support at one end and comprise, at the other end, a non-reactive group, e.g., a dideoxynucleotide incapable of incorporating further nucleotides. The short polynucleotide may also be a hairpin construct, provided that it does not interact with a polymerase.

In one embodiment of the present invention, each relatively long polynucleotide of the array comprises a hairpin loop structure, one end of which comprises a target polynucleotide, the other end comprising a relatively short polynucleotide capable of acting as a primer in a polymerase reaction. This ensures that the primer is able to perform its priming function during a polymerase-based sequencing procedure, and is not removed during any washing step in the procedure. The target polynucleotide is capable of being interrogated.

The term “hairpin loop structure” refers to a molecular stem and loop structure formed from the hybridisation of complementary polynucleotides that are covalently linked. The stem comprises the hybridised polynucleotides and the loop is the region that covalently links the two complementary polynucleotides. Anything from a 5 to 25 (or more) base pair double-stranded (duplex) region may be used to form the stem. In one embodiment, the structure may be formed from a single-stranded polynucleotide having complementary regions. The loop in this embodiment may be anything from 2 or more non-hybridised nucleotides. In a second embodiment, the structure is formed from two separate polynucleotides with complementary regions, the two polynucleotides being linked (and the loop being at least partially formed) by a linker moiety. The linker moiety forms a covalent attachment between the ends of the two polynucleotides. Linker moieties suitable for use in this embodiment will be apparent to the skilled person. For example, the linker moiety may be polyethylene glycol (PEG).

If the short molecules are polynucleotides in a hairpin construct, it is possible to ligate the relatively long polynucleotides to a minor proportion of the hairpins either prior to or after arraying the hairpins on the solid support.

The arrays have many applications in methods which rely on the detection of biological or chemical interactions with polynucleotides. For example, the arrays may be used to determine the properties or identities of cognate molecules. Typically, interaction of biological or chemical molecules with the arrays are carried out in solution.

In particular, the arrays may be used in conventional assays which rely on the detection of fluorescent labels to obtain information on the arrayed polynucleotides. The arrays are particularly suitable for use in multi-step assays where the loss of synchronisation in the steps was previously regarded as a limitation to the use of arrays. The arrays may be used in conventional techniques for obtaining genetic sequence information. Many of these techniques rely on the stepwise identification of suitably labelled nucleotides, referred to in U.S. Pat. No. 5,654,413 as “single base” sequencing methods.

In an embodiment of the invention, the sequence of a target polynucleotide is determined in a similar manner to that described in U.S. Pat. No. 5,654,413, by detecting the incorporation of nucleotides into the nascent strand through the detection of a fluorescent label attached to the incorporated nucleotide. The target polynucleotide is primed with a suitable primer (or prepared as a hairpin construct which will contain the primer as part of the hairpin), and the nascent chain is extended in a stepwise manner by the polymerase reaction. Each of the different nucleotides (A, T, G and C) incorporates a unique fluorophore at the 3′ position which acts as a blocking group to prevent uncontrolled polymerisation. The polymerase enzyme incorporates a nucleotide into the nascent chain complementary to the target, and the blocking group prevents further incorporation of nucleotides. The array surface is then cleared of unincorporated nucleotides and each incorporated nucleotide is “read” optically by a charge-coupled device using laser excitation and filters. The 3′-blocking group is then removed (deprotected), to expose the nascent chain for further nucleotide incorporation.

Because the array consists of distinct optically resolvable polynucleotides, each target polynucleotide will generate a series of distinct signals as the fluorescent events are detected. Details of the full sequence are then determined.

Other suitable sequencing procedures will be apparent to the skilled person. In particular, the sequencing method may rely on the degradation of the arrayed polynucleotides, the degradation products being characterised to determine the sequence.

An example of a suitable degradation technique is disclosed in WO-A-95/20053, whereby bases on a polynucleotide are removed sequentially, a predetermined number at a time, through the use of labelled adaptors specific for the bases, and a defined exonuclease cleavage.

A consequence of sequencing using non-destructive methods is that it is possible to form a spatially addressable array for further characterisation studies, and therefore non-destructive sequencing may be preferred. In this context, the term “spatially addressable” is used herein to describe how different molecules may be identified on the basis of their position on an array.

Once sequenced, the spatially addressed arrays may be used in a variety of procedures which require the characterisation of individual molecules from heterogeneous populations.

The following Examples illustrate the invention, with reference to the accompanying drawings.

### EXAMPLES

### Example 1

Glass slides were cleaned with decon 90 for 12 hours at room temperature prior to use, rinsed with water, EtOH and dried. A solution of glycidoxypropyltrimethoxysilane (0.5 mL) and mercaptopropyltrimethoxysilane (0.0005 mL) in acidified 95% EtOH (50 mL) was mixed for 5 min. The clean, dried slides were added to this mixture and left for 1 hour at room temperature rinsed with EtOH, dried and cured for 1 hour at 100° C. Maleimide modified DNA was prepared from a solution of amino-DNA (5′-Cy3-CtgCTgAAgCgTCggCAggT-heg-aminodT-heg-ACCTgCCgACgCT; SEQ ID NO:1) (10 μM, 100 μL) and N-[g-Maleimidobutryloxy]succinimide ester (GMBS); (Pierce) (1 mM) in DMF/diisopropylethylamine (DIPEA)/water (89/1/10) for 1 hour at room temperature. The excess cross-linker was removed using a size exclusion cartridge (NAP5) and the eluted DNA freeze-dried in aliquots and freshly diluted prior to use. An aliquot of the maleimide-GMBS-DNA (100 nM) was placed on the thiol surface in 50 mM potassium phosphate/1 mM EDTA (pH 7.6) and left for 12 hours at room temperature prior to washing with the same buffer.

The slide was inverted so that the chamber coverslip contacted the objective lens of an inverted microscope (Nikon TE200) via an immersion oil interface. A 60° fused silica dispersion prism was optically coupled to the back of the slide through a thin film of glycerol. Laser light was directed at the prism such that at the glass/sample interface it subtended an angle of approximately 68° to the normal of the slide and subsequently underwent Total Internal Reflection (TIR). Fluorescence from the surface produced by excitation with the surface specific evanescent wave generated by TIR was collected by the objective lens of the microscope and imaged onto an intensified charged coupled device (ICCD) camera (Pentamax, Princeton Instruments).

Images were recorded using a combination of a 532 Nd:YAG laser with a 580DF30 emission filter (Omega optics), with an exposure of 500 ms and maximum camera gain and a laser power of 50 mW at the prism.

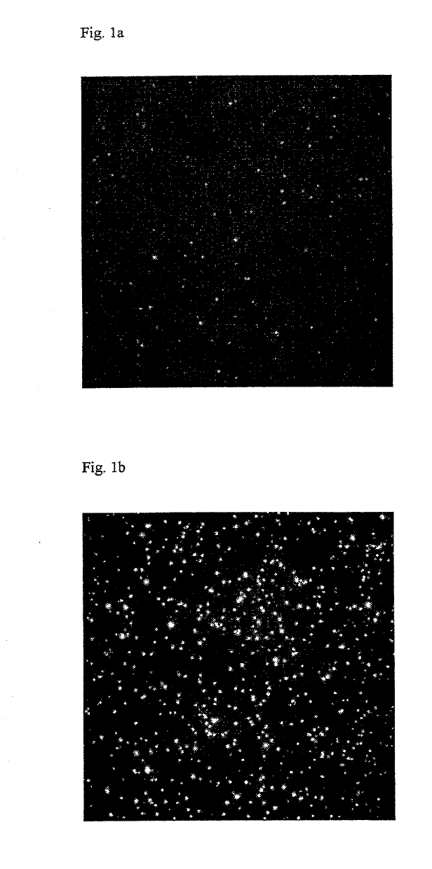

The presence of glycidoxypropyltrimethoxysilane gave improved results (FIG. 1a) compared to a control carried out in the absence of glycidoxypropyltrimethoxysilane.

### Example 2

Slides were cleaned with decon 90 for 12 hours prior to use and rinsed with water, EtOH and dried. A solution of tetraethoxysilane (0.7 mL) and N-(3-triethoxysilylpropyl)bromoacetamide (0.0007 mL) in acidified 95% EtOH (35 mL) was mixed for 5 minutes. The clean, dried slides were added to this mixture and left for 1 hour at room temperature, rinsed with EtOH, dried and cured for 1 hour at 100° C. Phosphorothioate modified DNA (5′-TMR-TACCgTCgACgTCgACgCTggCgAgCgTgCTgCggTTsTsTsTsT ACCgCAgCACgCTCgCCAgCg; SEQ ID NO:2) where s=phosphorothioate (100 pM, 100 μL) in sodium acetate (30 mM, pH 4.5) was added to the surface and left for 1 hour at room temperature. The slide was washed with a buffer containing 50 mM Tris/1 mM EDTA.

Imaging was performed as described in Example 1 and a good dispersion of single molecules was seen (FIG. 1b).

### Example 3

Slides were cleaned with decon 90 for 12 hours prior to use and rinsed with water, EtOH and dried. A solution of glycidoxypropyltrimethoxysilane (0.5 mL) in acidified 95% EtOH was prepared and the cleaned slides placed in the solution for 1 hour, rinsed with EtOH and dried. Amino modified DNA (5′-Cy3-CTgCTgAAgCgTCggCAggT-heg-aminodT-heg-ACCTgCCgACgCT; SEQ ID NO:1) (1 μM, 100 μL) was placed on the surface and left for 12 hours at room temperature. The slide was washed with a solution of 1 mM glycine at pH 9 for 1 hour and flushed with 50 mM potassium phosphate/1 mM EDTA (pH 7.6). A good dispersion of coupled single molecules was seen by TIR microscopy, as described in Example 1.

The slide was then exposed to a mixture containing Cy5-dUTP (20 μM) and T4 exo-polymerase (250 nM) and Tris (40 mM), NaCl (10 mM), MgCl₂ (4 mM), DTT (2 mM), potassium phosphate (1 mM), BSA (0.2 mgs/ml) 100 μL) at room temperature for 10 minutes and then flushed with Tris/EDTA buffer.

Imaging was performed using a pumped dye laser at 630 nm with a 670DF40 emission filter at 40 mW laser power using the TIR setup as described. A lower level of non-specific triphosphate binding was seen in the case using glycine, than in a control not treated with glycine.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A device comprising a high density array of a plurality of first molecules and a plurality of second polynucleotides immobilised on the surface of a solid support, wherein each molecule of at least a subset of said plurality of first molecules is shorter in length with respect to the distance from the planar surface of the solid support than the length of each of said second polynucleotide of at least a subset of said plurality of second polynucleotides such that said second polynucleotides are of a length and at a density that permits individual resolution of at least two of said second polynucleotides of said subset.

2. The device of claim 1, wherein said second polynucleotide comprises a first portion that corresponds in length with respect to the distance from the planar surface of the solid support to the length of said first molecule and a second portion that extends beyond the first portion and wherein said second portion is interrogated to individually resolve said second polynucleotides.

3. The device of claim 1, wherein each polynucleotide is immobilised by covalent bonding to the surface.

4. The device of claim 1, wherein the polynucleotides and the short molecules contain the same reactive group that attaches to the solid support.

5. The device of claims 1-4, wherein the polynucleotides are immobilised to the solid support via covalent attachment to an intermediate molecule and the short molecules are incapable of undergoing the same covalent attachment to the polynucleotides.

6. The device of claim 5, wherein the intermediate molecule and the short molecules are silane compounds.

7. The device of claims 1-4, wherein adjacent polynucleotides of the array are separated by a distance of at least 10 nm.

8. The device of claims 1-4, wherein the polynucleotides are separated by a distance of at least 100 nm.

9. The device of claims 1-4, wherein the polynucleotides are separated by a distance of at least 250 nm.

10. The device of claims 1-4, wherein the density of the polynucleotides is from 106 to 109 polynucleotides per cm².

11. The device of claims 1-4, wherein the density of the polynucleotides is from 10⁶ to 10⁸ molecules per cm².

12. The device of claims 1-4, wherein the relatively short molecules are polynucleotides.

13. A method of monitoring an interaction with a single polynucleotide, comprising resolving an arrayed polynucleotide with a device comprising a high density array of a plurality of first molecules and a plurality of second polynucleotides immobilised on the surface of a solid support, wherein each molecule of at least a subset of said plurality of first molecules is less in length with respect to the distance from the planar surface of the solid support than the length of each of said second polynucleotide of at least a subset of said plurality of second polynucleotides such that said second polynucleotides are of a length and at a density that permits individual resolution of at least two of said second polynucleotides of said subset.

14. A method for the production of an array of polynucleotides which are at a density that permits individual resolution, comprising arraying on the surface of a solid support, a mixture of relatively long polynucleotides and relatively short molecules, wherein the short molecules are in excess of the polynucleotides.

15. The method of claim 14, wherein the polynucleotides and the short molecules each have the same reactive group that attaches to the solid support or to an intermediate molecule.

16. The method of claim 14 or claim 15, wherein the polynucleotides and short molecules are brought into contact with the solid support in a single composition.

17. The method of claim 14 or claim 15, wherein the short molecules and the polynucleotides are arrayed separately, with the short molecules being brought into contact with the solid support first.

18. The method of claim 17, wherein a minor proportion of the arrayed short molecules comprise a functional group that reacts covalently with a functional group on the polynucleotides to enable the polynucleotides to be arrayed.

19. The method of claim 17, wherein, prior to being arrayed, a minor proportion of short molecules are modified in solution to provide the functional group complementary to that on the polynucleotides.

20. The method of claim 17, wherein the short molecules contain a functional group that is capable of reacting covalently with a complementary group on the polynucleotides, and wherein the polynucleotides are brought into contact with the solid support at a concentration that permits only a minor proportion of the short molecules to undergo reaction with the polynucleotides.

21. The method of claim 20, wherein those molecules that are not reacted with a polynucleotide are reacted with a capping agent.

22. The method of claim 21, wherein the capping agent is a relatively short polynucleotide.

23. The method of claim 14, wherein the relatively short molecules are polynucleotides, and both long and short polynucleotides are reacted with a functional group and then arrayed either directly onto the solid support or to an intermediate molecule bound to the solid support.

24. The method of claim 14, wherein the short molecules are polynucleotides in a hairpin construct, and the relatively long polynucleotides are ligated onto a minor proportion of the short molecules either prior to or after attachment of the short molecules to the solid support.


SEQUENCE LISTING (2 sequences)

SEQ ID NO: 1 - DNA, 33 bp, Artificial Sequence
  Synthetic Oligonucleotide
ctgctgaagcgtcggcaggtacctgccgacgct

SEQ ID NO: 2 - DNA, 61 bp, Artificial sequence
  Synthetic oligonucleotide
taccgtcgacgtcgacgctggcgagcgtgctgcggnnnntaccgcagcacgctcgccagcg